(19) United States
(12) Patent Application Publication
(10) Pub. No.: US 2009/0259121 A1
(43) Pub. Date: Oct. 15, 2009

(54) SYSTEM AND METHOD FOR CARDIOVASCULAR EXERCISE STRESS MRI

(75) Inventors: Orlando Paul Simonetti, Columbus, OH (US); Eric Lee Foster, Columbus, OH (US); John Wayne Arnold, New Philadelphia, OH (US); Subha V. Raman, Columbus, OH (US)

Correspondence Address: STANDLEY LAW GROUP LLP, 6300 Riverside Drive, Dublin, OH, 43017, US

(73) Assignee: The Ohio State University, Columbus, OH (US)

(21) Appl. No.: 12/424,835

(22) Filed: Apr. 16, 2009

Related U.S. Application Data
(63) Continuation of application No. PCT/US2007/081948, filed on Oct. 19, 2007.
(60) Provisional application No. 60/862,107, filed on Oct. 19, 2006.

(51) Int. Cl. A61B 5/055; A63B 22/02

(52) U.S. Cl. 600/410; 482/54

(57) ABSTRACT

A system and method for cardiovascular exercise stress magnetic resonance using a MRI-compatible treadmill and real-time imaging. The treadmill comprises non-ferromagnetic components so that it may be used in proximity to a MRI scanner. The treadmill is positioned adjacent to the MRI scan table. A treadmill control system is used to control the speed and grade of the treadmill to allow it to perform a wide range of exercise protocols. Patients complete an exercise protocol on the treadmill and are then moved to the MRI scan table. Images are acquired as quickly as possible post-exercise to more accurately diagnose cardiovascular disease in patients.

### CROSS-REFERENCE TO RELATED APPLICATIONS

This application is a continuation under 35 USC §120 of PCT/US2007/081948, filed Oct. 19, 2007, titled SYSTEM AND METHOD FOR CARDIOVASCULAR EXERCISE STRESS MRI, which is in turn entitled to benefit of a right of priority under 35 USC §119 from U.S. Ser. No. 60/862,107, filed Oct. 19, 2006, titled MAGNETIC RESONANCE COMPATIBLE TREADMILL, both of which are herein incorporated by reference in their entirety.

### TECHNICAL FIELD

Exemplary embodiments of the present invention relate to systems and methods for cardiovascular magnetic resonance imaging. More particularly, one exemplary embodiment of the present invention is a system and method for cardiovascular magnetic resonance imaging using exercise stress.

### BACKGROUND OF THE INVENTION

Since it was first proposed as a diagnostic tool for angina almost 75 years ago, treadmill exercise stress testing quickly became, and still remains, an essential tool in the detection and treatment of heart disease. The Bruce Treadmill Test, first published in 1963, is the most commonly used exercise test protocol in the United States, and has been shown to have high diagnostic and prognostic value. The addition of imaging to the exercise stress test further improves sensitivity and specificity, providing greater diagnostic accuracy than exercise ECG alone. According to some estimates, over 10 million stress studies are performed in the US each year in conjunction with nuclear or echocardiographic imaging. The systems currently available for both types of exercise stress imaging tests reflect their respective techniques and limitations.

With nuclear stress testing, a radioisotope dose is injected via a peripheral vein prior to rest imaging and another radioisotope dose is injected prior to stress imaging. A 15-minute delay is required after each injection to allow sufficient extraction by heart muscle (myocardial) cells to occur for subsequent detection of myocardial perfusion with a gamma camera. Each nuclear imaging step takes 25 minutes, assuming sufficient detectable counts; acquisition times may be longer in obese patients. If there is significant patient motion at any time during the acquisition obscuring reconstructed image quality, the acquisition must be repeated. Cardiac nuclear images are also affected by adjacent gut uptake of the radioisotope. If this interference is seen after images are acquired, the patient is asked to drink water or eat a fatty snack to allow bowel contraction away from the heart, and then the acquisition is repeated. This may involve an additional 1-2 hour delay to the overall test time, requiring that a patient scheduled for a stress test at a nuclear imaging facility allow 4 to 6 hours for test completion.

A further limitation of nuclear stress perfusion imaging is spatial resolution, which is on the order of 1 cm. This reduces the specificity of abnormal findings, which may be due to attenuation artifact rather than heart disease. This also reduces sensitivity of an abnormal test result, which is not uncommon in patients with “balanced” ischemia that prevents recognition of a focal perfusion abnormality. False positive tests lead to further unnecessary and usually invasive testing such as cardiac catheterization, while false negative tests may allow a condition to go undiagnosed until a catastrophic event such as a heart attack occurs making the diagnosis obvious. A final limitation to nuclear testing is that it requires injection of a radioisotope into the body in an era when both providers as well as consumers of health care seek to minimize risk in medical testing.

Stress echocardiography is the other system currently used for stress imaging, and involves acquisition of cardiac images at rest and stress using an ultrasound transducer placed over various locations on the chest wall. Its appeal over nuclear stress testing has been that it does not use ionizing radiation. It is limited, however, in its signal-to-noise ratio. Further limitations occur due to the time required to manually locate the cardiac imaging planes which may be particularly challenging in a patient who is breathing heavily after submaximal exercise. Patient breath-holding is required due to the small field-of-view and large extent of motion of the heart with heavy breathing after exercise. Any additional time introduced between peak stress and successful image acquisition reduces test accuracy because cardiac function abnormalities caused by ischemia quickly resolve after exercise has terminated; thus, the resulting cardiac images do not necessarily reflect the heart's activity at peak stress. If the chest wall is expanded due to obesity or additional airspace between the transducer and the heart due to various forms of chronic lung disease, the frequent limitation of “acoustic window” in ultrasound-based cardiac stress imaging results.

The unmet clinical and technical needs for accurate stress cardiac imaging include: (1) high-resolution cardiac imaging that is impervious to artifact imposed by nuclear attenuation or acoustic window; (2) rapid image acquisition to accurately reflect cardiac performance at peak stress; and (3) elimination of ionizing radiation. Magnetic resonance imaging (MRI)-based stress testing should be able to meet these needs, but requires considerable advances in treadmill design, hydraulics, patient localization, monitoring, and imaging software over the inadequate MRI-based solutions proposed to date.

Current MRI-based stress testing requires pharmacologic stress, but has demonstrated success and superior accuracy when compared to stress echocardiography and nuclear imaging. Pharmacological stress has remained the only practical approach to MRI stress imaging due to the lack of MRI-compatible exercise and monitoring equipment. However, exercise is preferable to pharmacologic stress testing because it links physical activity to symptoms and imaging findings. While upright treadmill exercise is the physiologically preferred method of cardiovascular stress testing, it presents significant challenges for use with MRI. Treadmills are typically powered by electromagnetic motors (e.g., to move the treadmill belt and control its elevation) and contain a multitude of ferromagnetic parts and components, precluding their use in close proximity to a MRI magnet. Ferromagnetic materials are typically used to provide structural strength at low cost but they are unsuitable for use with MRI.

Rerkpattanapipat and his team at Wake Forest University [1] have investigated the possibility of combining treadmill stress with MRI imaging. A treadmill for exercising patients was located outside of the MRI room. Safe positioning of the exercise and monitoring equipment required the patient to walk about 20 feet from the treadmill to the MRI system. With the use of segmented imaging sequences requiring breath-holding, they showed that multi-slice cine images of cardiac function could be completed within 60-90 seconds post-exercise. The sensitivity and specificity to detect >70% coronary artery diameter narrowing in 27 patients were 79% and 85%, respectively. Although the feasibility of detecting severe coronary artery stenosis by exercise stress MRI using a treadmill positioned outside the magnet room was demonstrated by Rerkpattanapipat, the concept has been insufficient for clinical use elsewhere because of the problems associated with moving a patient from outside of the room to the MRI system quickly and safely after reaching maximal cardiovascular stress.

Another type of exercise device that has been used for stress testing is the bicycle ergometer. A supine bicycle ergometer that allows imaging during exercise inside a closed-bore magnet has been offered by Lode BV (the Netherlands) [2] as a commercial product. However, pedaling in a totally supine position is uncomfortable and exercise time is limited by the onset of leg fatigue. Depending on the height of the patient and the size of the magnet bore, there may be insufficient knee-to-bore clearance while cycling. This supine ergometer has been primarily used in research studies that did not require maximal exercise. For example, Niezen et al. [3] performed measurements of aortic and pulmonary flow at two levels of submaximal exercise in 16 healthy volunteers. Even within this group of healthy patients exercising at relatively low workloads, one patient could not complete the protocol because of muscle fatigue. This problem may be compounded in patients with known or suspected heart disease.

Although bicycle ergometry appears suitable for blood flow studies during submaximal exercise, treadmill exercise is preferred to bicycle ergometry for cardiovascular stress testing in the United States. Fatigue of the quadriceps muscles in patients who are not experienced bicyclists is a limitation to achieving target heart rate. Untrained patients typically achieve only 80-90% of their treadmill maximum oxygen consumption on a bicycle ergometer. In addition, bicycle ergometry requires cooperation of the patient to maintain pedal speed at the desired level.

Attempts at exercise MRI using both devices have had limited success due to technical and physiologic challenges. Despite tremendous advances in cardiovascular testing in the last few decades, consistently accurate stress imaging remains an important target for technology development to reduce uncertainty in the diagnosis and treatment of patients with many forms of cardiovascular disease. Cardiac Magnetic Resonance (CMR) already provides, in a single examination, high resolution assessment of stress wall motion, stress perfusion, and myocardial viability. The combination of exercise stress testing and CMR may have a significant impact on the clinical diagnosis and treatment of cardiovascular disease.

There is a need for a treadmill that could be used in a MRI room to allow it to function much like a standard exercise stress lab. In addition, there is a need for a treadmill that can be positioned close to the MRI patient table to minimize the time from exercise to imaging. Time delays are important, because function imaging must be completed within 60 seconds post-exercise to capture cardiac wall motion abnormalities induced by exercise. Finally, there is a need for a treadmill that can be positioned close to the MRI patient table to minimize the traveling distance immediately following maximal exercise to increase patient safety, especially for those patients who are less-mobile or de-conditioned cardiac patients.

### SUMMARY OF THE INVENTION

Exemplary embodiments of the present invention include a system and method for cardiovascular exercise stress magnetic resonance using a MRI-compatible treadmill and real-time imaging. The MRI-compatible treadmill is positioned directly adjacent to the MRI scan table, enabling configuration of the MRI scan room similar to a stress-echocardiography lab. The configuration allows for exercise CMR that is safe and successful in typical patients requiring cardiac stress testing. A MRI-compatible treadmill allows for the patient to dismount the treadmill and to move immediately onto the scanner table. This approach allows acquisition of images as quickly as possible post-exercise in order to capture transient exercise-induced wall motion abnormalities (WMAs) that can rapidly resolve after ischemia is reversed. The persistence of WMAs is most likely related to the severity of coronary artery disease (CAD) (number of vessels involved, percent stenosis), the presence of coronary collateral flow, and the duration of ischemia. Therefore, in order to accurately diagnose patients with less severe single-vessel CAD, associated with a high ischemic threshold and rapid WMA resolution, imaging performed as close as possible to peak exercise, and ideally with no delay is preferable. Minimizing the time between the end of exercise and the beginning of imaging maximizes the sensitivity of the test.

An exemplary embodiment of the present invention may be an improvement over prior art approaches that require the patient to walk from a treadmill to the MRI table. A treadmill positioned any distance from the MRI table, whether inside or outside the room, creates a potential safety concern. Immediately following maximal exercise patients may become dizzy and lightheaded and subject to falling. With an exemplary embodiment of the present invention, the exercise stress CMR test may be conducted safely by positioning the treadmill immediately adjacent to the MRI table, as it is in exercise stress echocardiography.

The use of MRI-compatible equipment that allows the positioning of the exercise stress system immediately adjacent to the MRI system, and the use of rapid real-time imaging techniques eliminating breath-hold requirements allows exercise stress MRI to be successfully performed in cardiac patients, and has the potential to achieve higher levels of diagnostic accuracy than previously shown for MRI or other stress imaging modalities.

### BRIEF DESCRIPTION OF THE DRAWINGS

FIG. 1 is an illustration of components and the configuration of components for a MRI-compatible treadmill system according to an example embodiment of the present invention.

FIG. 2 is an equipment layout and a schematic diagram of an electric motor driven pump for powering a treadmill mounted hydraulic motor through hoses according to an example embodiment of the present invention.

FIG. 3 is a diagram of a stainless steel hydraulic motor for a MRI-compatible treadmill according to an example embodiment of the present invention.

FIG. 4 is a diagram of a hydraulic drive and elevation system for a MRI-compatible treadmill according to an example embodiment of the present invention.

FIG. 5 is a schematic diagram of hydraulic components for a MRI-compatible treadmill according to an example embodiment of the present invention.

FIG. 6 is a screen shot of a computer for a treadmill control system according to an example embodiment of the present invention.

FIG. 7 is a configuration diagram for patient positioning equipment according to an example embodiment of the present invention.

FIG. 8 is an exercise CMR protocol according to an example embodiment of the present invention.

### DETAILED DESCRIPTION OF EXAMPLE EMBODIMENTS

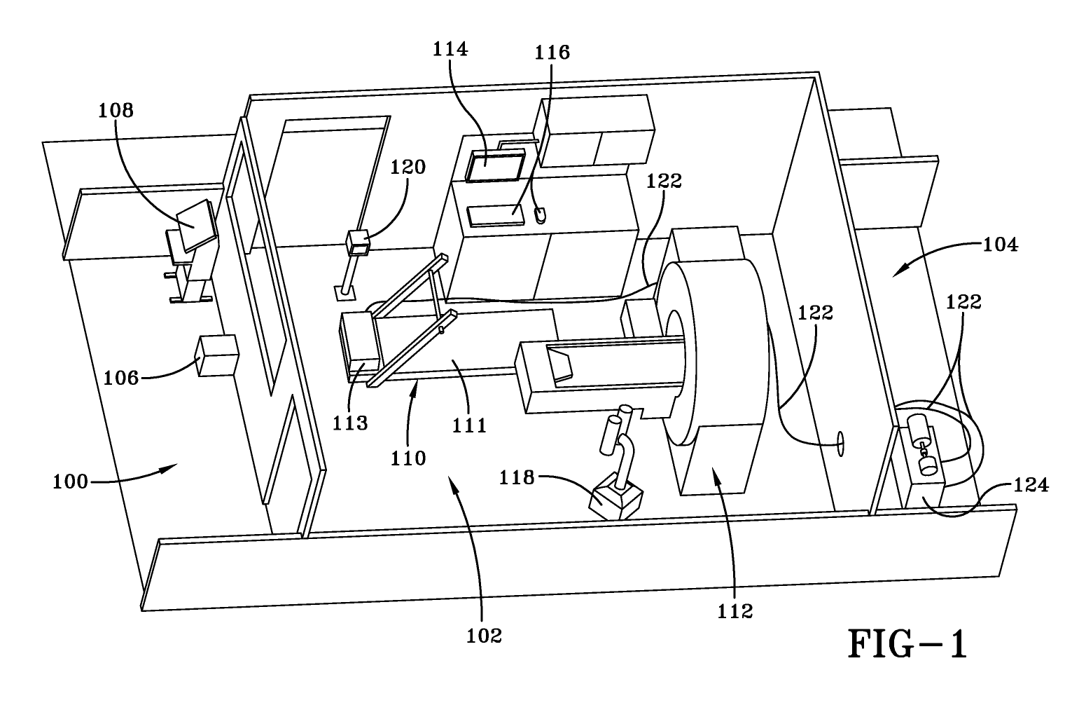

Referring to FIG. 1, an illustration of components and the configuration of components for a MRI-compatible treadmill system according to an example embodiment of the present invention is shown. In this example, components of the present invention are contained in a control room 100, a scan room 102 and an equipment room 104. One or more computers 106 in the control room 100 support control and monitoring of components in the scan room 102. A treadmill control system computer may be used to communicate with the treadmill 110. Another scanner computer may be used to communicate with the MRI scanner 112. The scanner computer may be used to control functionality of the MRI scanner 112 related to data acquisition, image reconstruction, and image display and analysis.

A hydraulic powered treadmill 110 and MRI scanner 112 are contained in the scan room 102. The hydraulic powered treadmill 110 is connected to a hydraulic power pack 124 via hydraulic hoses 122. In FIG. 1, certain hose segments are shown as including only one hose for clarity purposes. Thus, it should be recognized that there may be more than one hose in any of the hose segments as needed. The hydraulic power pack 124, which may contain ferromagnetic components, is contained in a separate equipment room 104. In other exemplary embodiments, a hydraulic power pack may be located in any suitable location including, but not limited to a room, such that the hydraulic power pack is not adversely affected by a MRI scanner. A wireless monitor 114 and wireless keyboard/mouse 116 may be used in the scan room 102 to control the components housed in the scan room 102. In other exemplary embodiments, a monitor and keyboard/mouse, each of which may or may not be wireless, may be situated in any other suitable location, including outside of a scan room, to allow effective control of any desired components.

Because the treadmill 110 is fully MR compatible, it may be placed immediately adjacent to the MRI scanner 112 or in any desired position within the scan room 102. Complete MR compatibility also allows for the use of the treadmill with higher field strength magnets, i.e., 3.0 Tesla. The resulting configuration of the scan room 102 may be very similar to the setup for a standard exercise echocardiograph lab.

An exemplary embodiment of a treadmill 110 may comprise a support and a belt 111 rotatably associated with the support. Furthermore, an exemplary embodiment of a MR-compatible treadmill comprises programmable components so that it may be controlled independently through a standard PC as well as with leading treadmill stress testing software. The programmable components may execute any of a variety of exercise protocols, including the standard Bruce Treadmill Exercise protocol. The Bruce Treadmill Exercise protocol automatically advances a patient through set stages of speed and elevation as shown in Table 1. The treadmill may also or alternatively be controlled manually.

TABLE 1
Standard Bruce Exercise Protocol for Stress Testing

| Stage | Speed (mph) | Grade (%) |
|---|---|---|
| 1 | 1.7 | 10 |
| 2 | 2.5 | 12 |
| 3 | 3.4 | 14 |
| 4 | 4.2 | 16 |
| 5 | 5.0 | 18 |
| 6 | 5.5 | 20 |
| 7 | 6.0 | 22 |

One primary challenge in MRI-compatible treadmill design is the drive system. Traditional electrical motors use ferromagnetic components with significant mass and can pose a severe hazard if brought into close proximity to the MRI magnet. By its nature, an electromagnetic motor cannot be made non-magnetic. For this reason, in an example embodiment of the present invention, a totally non-ferromagnetic hydraulic motor is used to power the treadmill. The treadmill 110 may be comprised of components made from non-ferromagnetic materials including, but not limited to, stainless steel or aluminum such that it may effectively operate in close proximity to the MRI scanner 112. Material choices may depend on tradeoffs between the necessary strength of the material compared to the increased cost of using stainless steel, for example. The treadmill 110 is constructed without using electric motors to directly power either the treadmill belt or the elevation mechanism. This approach is similar to the design strategy employed in underwater treadmills used for aqua-therapy such as those described in U.S. Pat. Nos. 5,558,604, 5,921,892, and 6,857,990.

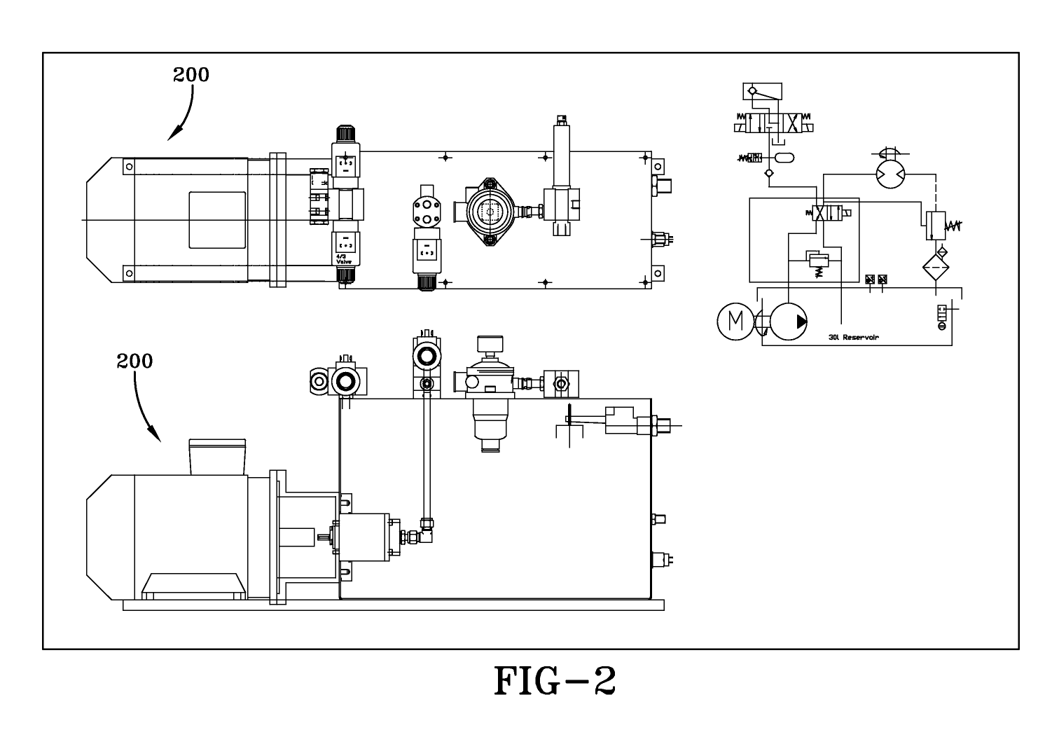

In this example, hydraulic power pack 124, comprising an electrical motor driven pump 200 as shown in FIG. 2, is located in an equipment room 104 outside the scan room 102. The pump forces hydraulic fluid from a reservoir into hydraulic hoses 122 that carry the pressurized fluid into the scan room 102 to a non-ferromagnetic hydraulic motor 113, which may be mounted on the front of the treadmill 110 or in another suitable location for effective operation, including locations directly on and not directly on treadmill 110.

A patient completes all or part of an exercise protocol on the treadmill and then moves to the MRI scanner. Medical staff, which may be present in the room during the stress test, may assist the patient in transferring from the treadmill to the MRI scanner. The lift system of the treadmill may be used to assist in transferring the patient by positioning the height of the treadmill to allow easy transfer of the patient to the MRI table. Because the treadmill may be placed directly adjacent to the MRI scanner 112, some patients may not require assistance while transferring from the treadmill 110 to the MRI table. Cardiac imaging data is collected and analyzed following the stress test so that the presence or extent of cardiovascular disease may be determined.

FIG. 1 further shows patient monitoring equipment according to an example embodiment of the present invention. Continuous 12-lead ECG monitoring of the patient may be used during the exercise test. A standard 12-lead ECG system 108 may be used by positioning it at the entrance to the MRI scan room 102, close enough to monitor the patient both on the treadmill 110 and on the MRI patient table when the patient is outside of the magnet bore. Alternatively, the unit may also be positioned in the adjacent control room 100 (as shown), with cabling run through the wall into the MRI scan room 102 to the patient on the treadmill 110. It would also be feasible to implement a MRI compatible 12-lead ECG system for use within the magnet room itself, although no such device is currently commercially available. While inside the bore, the ECG is non-diagnostic due to magneto-hydrodynamic artifacts caused by blood flow within the magnetic field. However, heart rate and rhythm may be monitored continuously with a wireless ECG unit. In an example embodiment of the present invention, the unit may be provided by MRI manufacturer Siemens Medical Solutions, Malvern, Pa. Other MRI-compatible wireless ECG systems are commercially available. This setup allows medical staff to quickly disconnect the patient from the 12-lead ECG system after exercise, while continuing to monitor heart rate. MRI-compatible manual and automatic non-invasive blood pressure equipment 120 such as that from Medrad, Inc., Pittsburgh, Pa. may be used to monitor blood pressure before, during, and after the stress test.

The MRI scanner 112 is controlled via a MRI-compatible in-room console such as one available from Siemens Medical Solutions, Malvern, Pa., and a start button located on the front panel of the magnet housing. The in-room console, designed primarily for interventional MRI applications, duplicates the functionality of the main imaging console. A power contrast injector 118 such as one from Medrad Corp., Pittsburgh, Pa. may be outfitted with a manual control switch for operation from within the MRI scan room 102. The injection protocol may be pre-programmed and loaded so that it can be executed immediately at the start of the perfusion scan from within the MRI room. In an example embodiment of the present invention, all equipment necessary to conduct the treadmill exercise test with continuous ECG 108 and blood pressure monitoring 120, as well as the equipment necessary to control the MRI procedure, is positioned to allow the test to be performed within the MRI scan room 102. As a result, the stress testing team is able to remain in the room and in direct communication with the patient at all times.

FIG. 2 is an equipment layout and a schematic diagram of an electric motor driven pump 200 for powering a treadmill mounted hydraulic motor through hoses according to an example embodiment of the present invention. One example of an electric motor driven pump 200 is commercially available from The Water Hydraulics Co. Ltd.

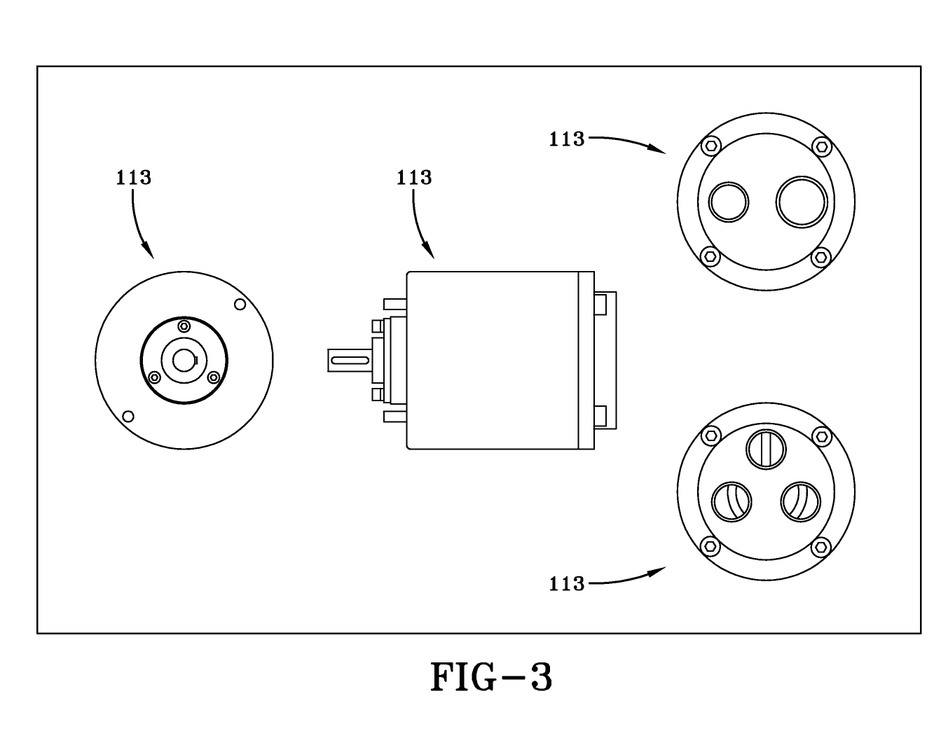

In this example, the flow of hydraulic fluid from power pack 124 powers hydraulic motor 113, which may be comprised of stainless steel. Hydraulic motor 113 turns a drive shaft. An example of a hydraulic motor 113 is shown in FIG. 3, which is one embodiment that may be commercially available from The Water Hydraulics Co. Ltd. Referring to the example of FIG. 3, a diagram is shown of a stainless steel hydraulic motor 113 that may be used in association with an exemplary embodiment of a MRI-compatible treadmill.

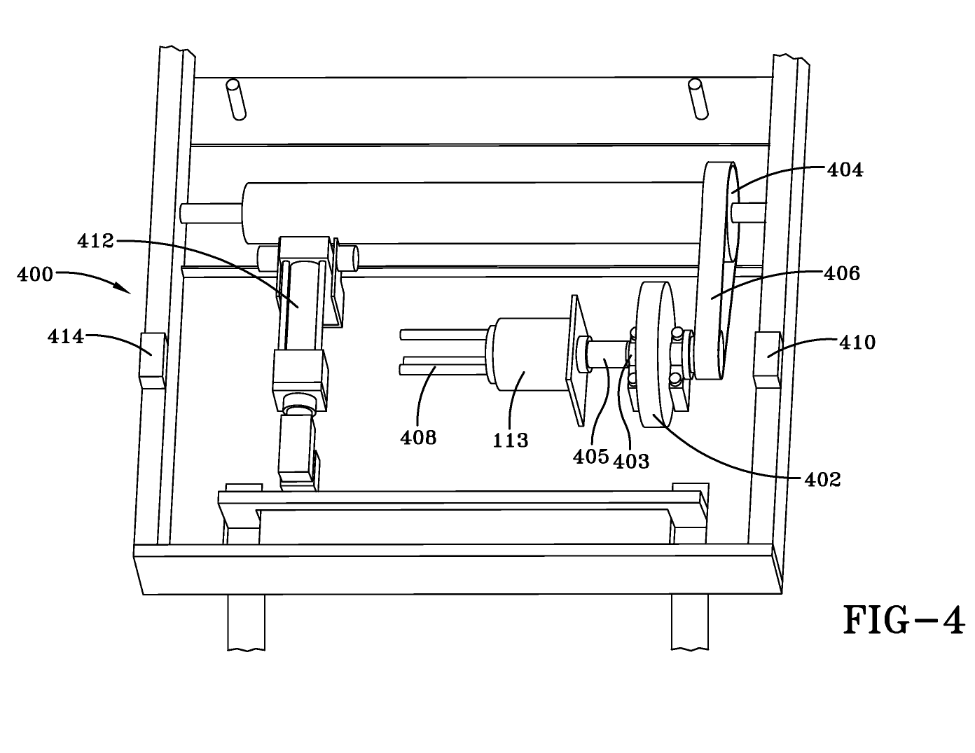

Referring to FIG. 4, a diagram of a hydraulic drive and elevation system 400 for a MRI-compatible treadmill according to an example embodiment of the present invention is shown (without a cover for purposes of clarity). An example of a hydraulic drive and elevation system 400 may be comprised of non-ferromagnetic materials including, but not limited to, stainless steel, such that it may not be adversely affected by a MRI scanner 112 when in close proximity. In this example, a non-ferromagnetic flywheel 402 attached to a driveshaft 403 (via shaft coupler 405) may attenuate inertial differences during footplant and speed change. The flywheel 402 is connected to a drive roller 404 with a belt 406. Drive roller 404 may serve as a support for belt 111, whereby the drive roller 404 may be adapted to cause rotation of belt 111 and belt 111 may be adapted to rotate about drive roller 404. There may be at least one drive roller to facilitate desired rotation of belt 111. Hoses 408 include at least one return hose and at least one input hose. A return hose 408 cycles the hydraulic fluid back to the reservoir. In this exemplary embodiment, hoses 408 are attached to the treadmill via MR-compatible, hydraulic quick couplings to allow for quick, clean setup and teardown.

In an exemplary embodiment, a treadmill design may use basic fluid power components currently available in industrial applications. However, in other exemplary embodiments, certain components of a treadmill including, but not limited to, the fluid power components may not be “off-the-shelf” and may be custom built for a particular application according to specifications.

An example of the operation of one embodiment will now be described. The power flow starts with the prime mover. With regard to an exemplary embodiment of a power pack 124, a variable speed electric motor supplies the power to control the treadmill belt speed. In one example of a motor driven hydraulic pump 200 of power pack 124 such as shown in FIG. 2, power from the electric motor is supplied via a shaft and flexible coupler to a fixed displacement hydraulic pump. The power is converted to flow proportional to rotational speed and pressure proportional to the treadmill load. The fixed displacement hydraulic motor 113, which may be connected to the treadmill belt by appropriate pulleys and belt 406, converts the fluid power to rotational power. In an exemplary embodiment, the pump output flow may connect directly to the motor inlet. The speed of the hydraulic motor may be virtually proportional to the electric motor speed in one exemplary operation. In this example, the speed relationship approximates a direct proportion, but the relationship is non-linear since internal leakage rates are dependent on load, temperature and other factors. In an exemplary embodiment, a resulting drive ratio variability may necessitate the use of a feedback control designed to maintain speed independent of load and other factors. Treadmill speed feedback is provided by an optical sensor 410 mounted adjacent to the flywheel 402. Other suitable speed sensing systems may be employed. The motor outlet flows through a hydraulic braking valve 502 capable of maintaining the appropriate pressure on the motor outlet to control speed and prevent inlet cavitation when operated at high gradients.

In further description of one example of an operation of one embodiment, the Bruce Treadmill Exercise protocol requires the treadmill to attain a maximum grade of 22% to accommodate patients with a wide range of physical conditions. The treadmill grade (gradient) is controlled by an ancillary circuit mounted on the power pack 124. An accumulator 500 of power pack 124 is charged with a volume of fluid sufficient to operate the treadmill 110 for a complete patient test session. At each protocol stage, a portion of the stored fluid is directed to the non-ferromagnetic treadmill lift cylinder 412 by way of valves and conductors (hoses in communication with cylinder 412 are not shown in FIG. 4 for clarity purposes). As one example of an alternative, a conventional linear actuator may be positioned on the hydraulic power pack outside of the MRI room. The standard hydraulic cylinder is actuated by the linear motor and its movement replicated by the MR-compatible cylinder via hydrostatic transmission. Elevation feedback may be provided using either a linear position sensor mounted on the cylinder, or a fluid-filled tilt sensor mounted to the treadmill frame as listed in Table 2.

The remaining design elements of an exemplary system include components suitable for a safe, reliable machine. With particular reference to this example, a pressure relief valve is installed at the pump outlet. Non-ferromagnetic hoses and couplers comprise the required fluid conductors. The couplers are sized and polarized to prevent incorrect connection during equipment setup. System cooling is provided by the reservoir. Filtration is built into the power pack 124 to filter the fluid returning from the circuits as well as fluid added to the system. Level sensors and pressure switches are used to complete the control circuits.

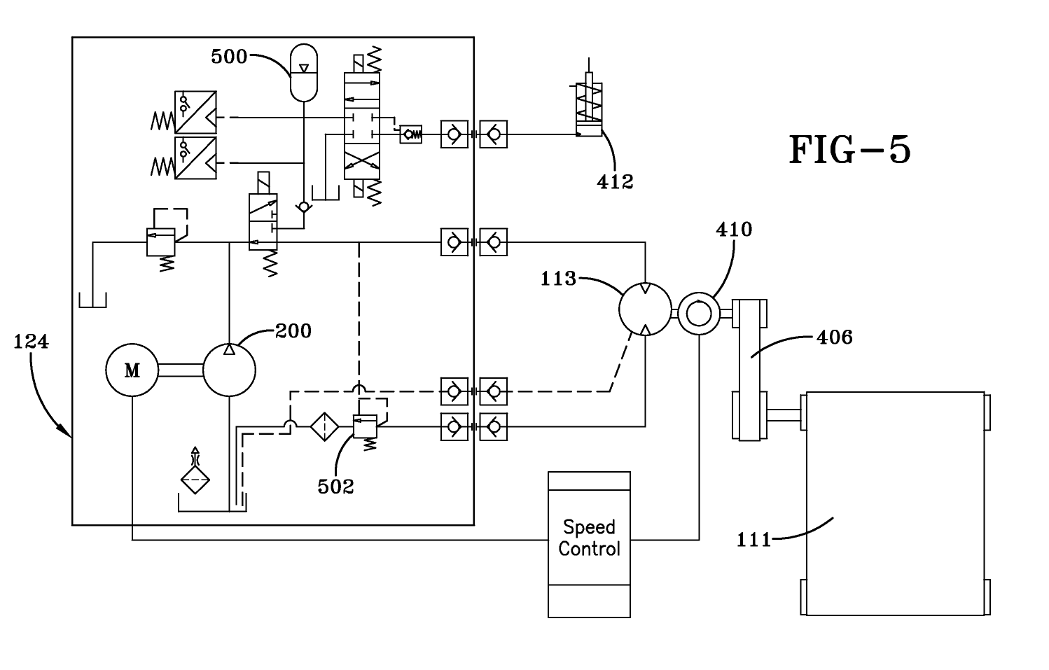

Referring to FIG. 5, a schematic diagram of hydraulic components for a MRI-compatible treadmill according to an example embodiment of the present invention is shown. Due to the placement of the treadmill system within a healthcare facility, the hydraulic power system may be designed to use water rather than traditional oil-based hydraulic fluids. Water based hydraulic fluids allow for simple cleanup of any accidental fluid leakage from the system as well as eliminate the danger of combustion of the hydraulic fluid. It also makes the system more universal by eliminating the need for on-hand stock of hydraulic fluid. Furthermore, with reference to FIG. 5, power pack 124 may include a braking valve 502 to help control belt speed.

In this example, the treadmill control system is located in the control room 100 outside the scan room 102 as shown in FIG. 1. An application executing on a control computer 106 communicates with the programmable components of the treadmill 110 to control the speed and grade of the treadmill 110. The control program of computer 106 flexibly and automatically runs the treadmill speed and elevation through a preset exercise protocol such as the Bruce Treadmill Exercise protocol or any other exercise stress protocol. The control program allows for feedback control to ensure the protocol is being followed precisely. An optical sensor 410 positioned adjacent to the flywheel 402 monitors the speed and sends a signal back to the controller. An angle sensor 414 mounted on the support provides elevation feedback. For safety purposes, a manual control emergency stop button may be located on the treadmill. Additional sensors shown in Table 2 may further provide for safe operation of the hydraulic treadmill system.

TABLE 2
MRI Compatible Treadmill Control System Sensor Options

| SENSORS | TYPE | LOCATION |
|---|---|---|
| Functional | | |
| Belt speed | Optical sensor with signal | Located on treadmill |
| | carried on fiber optic cable | at flywheel |
| Elevation | Option 1: | On elevation cylinder |
| | Linear potentiometer | |
| | Option 2: | Any flat surface under |
| | Fluid filled tilt sensor | hood of treadmill |
| Motor pressure | Pilot operated brake valve | Motor outlet with |
| control | | sensing from motor inlet |
| Safety | | |
| Emergency Stop | Button switch | Handle of treadmill |
| Water level | Level | Reservoir |
| Temperature | Temperature switch | Reservoir |

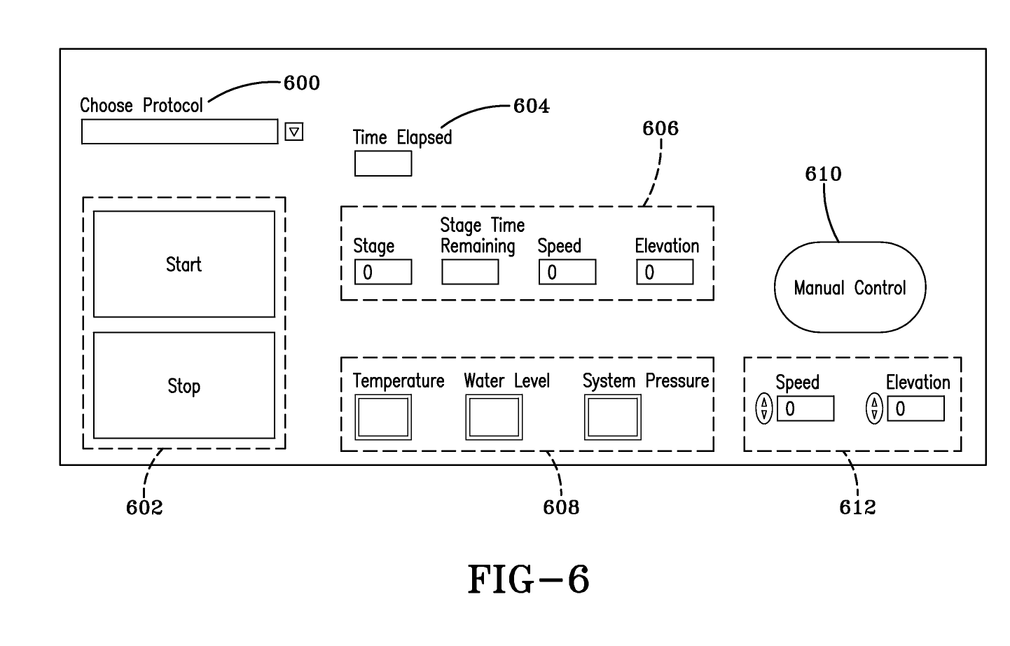

Referring to FIG. 6, a screen shot of an application for a treadmill control system according to an example embodiment of the present invention is shown. To control the operation of the treadmill both manually 610 and through a prescribed exercise protocol 600, the hydraulic motor speed and treadmill elevation 612 are input from a computer screen. Start and stop options 602 are used to start and end a selected protocol 600. The screen also shows the time elapsed 604, current stage 606 as well as time remaining in the stage and the speed and elevation for the stage. Status information 608 related to temperature, water level, and system pressure for the hydraulic system is also communicated on the screen.

A speed signal is routed to a motor controller that controls the speed of the electric motor located in the power pack outside the MRI exam room. The electric motor controls the speed of a pump, which in turn controls the rate of fluid flow delivered to the hydraulic motor located on the treadmill. The signal from the motor shaft speed sensor is fed into a feedback loop where it is compared with the intended hydraulic motor speed. A signal is sent to the electric motor control, which alters the speed of the electric motor.

At higher treadmill elevations, depending mainly on the weight of the treadmill user, the work of the person running on the treadmill acts to drive the motor to a higher speed that is not controlled by the pump and electric motor. At this point, the motor brake valve is activated, creating back pressure to the hydraulic motor. The hydraulic motor then acts as a brake, enabling the system to maintain the prescribed speed. An emergency stop button located on the body of the treadmill provides a motor shutoff signal if needed.

The treadmill elevation signal is output to the elevation mechanism. The mechanism may be a pre-charged accumulator that outputs the desired quantity of fluid through a valve either to a non-ferromagnetic hydraulic cylinder or to a master-slave cylinder system in which a traditional hydraulic cylinder located outside the room controls a slave cylinder located on the treadmill. A feedback signal is received from the elevation sensor, which may be either from a linear potentiometer located on the elevation cylinder or a fluid filled tilt sensor located on any flat surface of the treadmill. This signal enters a separate feedback loop where it is compared to the intended elevation.

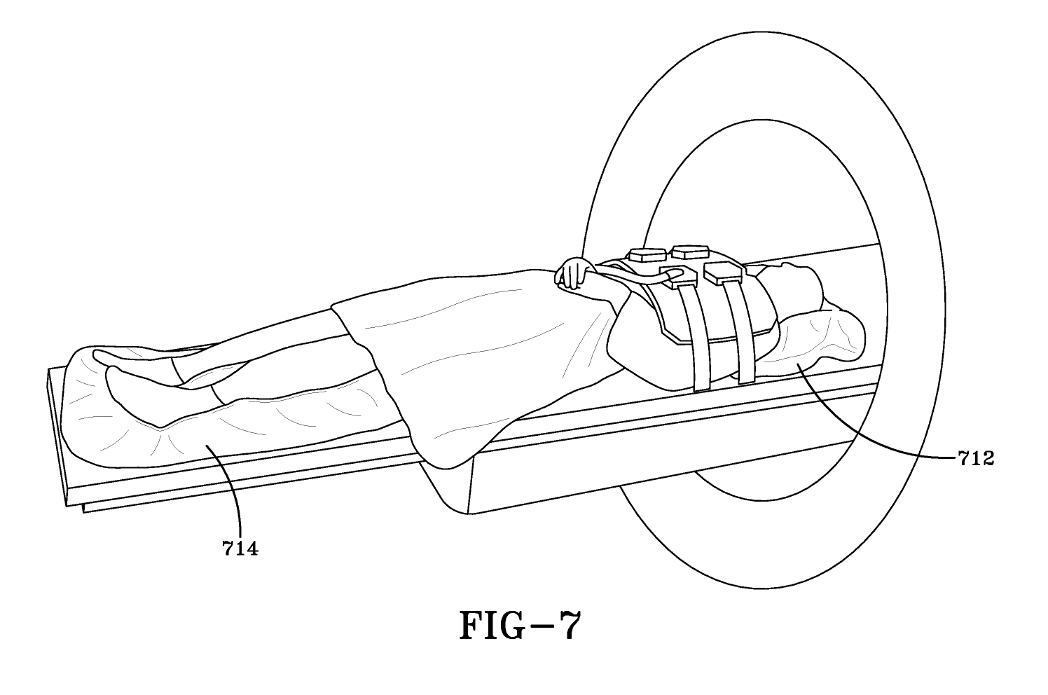

Referring to FIG. 7, a configuration diagram for patient positioning equipment according to an example embodiment of the present invention is shown. Before exercise, a patient is positioned on the MRI table using two vacuum mattresses 712, 714 such as those available from Vac-Lok Cushions, MEDTEC, Orange City, Iowa, and slice localization and resting function scans are performed. One vacuum mattress is placed under the head and shoulders 712 and the other under the legs extending from foot to upper thigh 714. Removal of air with a vacuum pump causes the mattresses to rigidly conform to the body. These devices are commonly used for repositioning of patients undergoing repeated radiation therapy sessions. This system ensures that the patient returns to the same position after exercise such that stress imaging may be performed using the slice planes previously prescribed at rest.

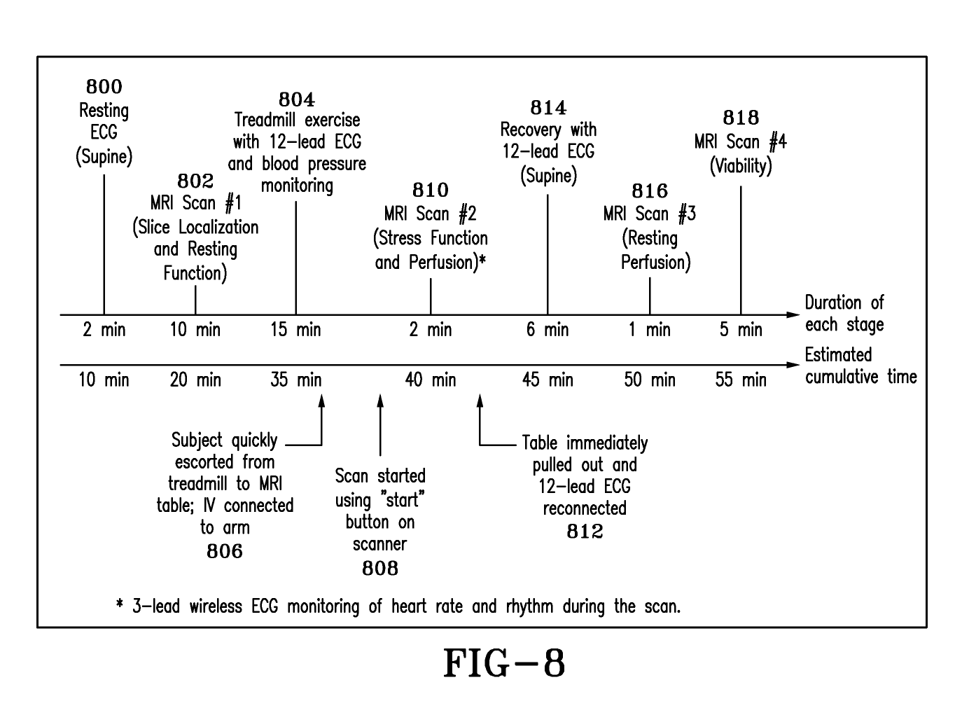

Referring to FIG. 8, an exercise CMR protocol according to an example embodiment of the present invention is shown. Patient preparation includes insertion of an intravenous (IV) needle and the standard placement of both the 12-lead and the wireless ECG electrodes on the chest. Supine 12-lead ECG and blood pressure (BP) are recorded at rest 800. The supine resting ECG is used for direct comparison with the supine recovery ECG post-exercise. Next, patients are positioned on the MRI table using the vacuum mattresses. Air is removed from the mattresses through a vacuum line located inside the MRI room.

In an example embodiment of the present invention, slice localization by single-shot steady-state free precession (SSFP) imaging is followed by resting cine imaging 802. The cine function sequence is configured to scan each slice position for approximately 2 seconds, while the temporal resolution varies depending on the size of the patient and the resulting field of view. A test acquisition for first-pass perfusion may be performed without contrast agent. Pulse sequences are queued for stress imaging such that they may be executed automatically from the scan start button located on the magnet. The patient is then removed from the magnet. Certain makes and models of MRI scanners may require medical staff to use extra care when removing the patient so as not to pull the table all the way out of the magnet, and not to move the surface array coil too drastically. Either of these actions may cause certain systems to repeat adjustments prior to the start of the stress scan, causing delays.

Next, the patient exercises on a treadmill positioned inside the MRI room 704. In an example test, the treadmill speed and elevation are progressively increased every three minutes following the standard Bruce protocol. 12-lead ECG is continuously monitored during exercise. Blood pressure is measured and a hard copy of the ECG is obtained at the midpoint of each Bruce protocol stage. As with conventional stress testing, patients are continuously monitored by a nurse and/or physician who may stop the test at any time based on recognition of adverse endpoints or in response to the patient's request.

After reaching his or her exercise limit or the maximum predicted heart rate (MPHR) based on age (220−age), the patient is quickly escorted to the MRI table 806. The surface coil is placed on the chest, the contrast injector is connected to a previously inserted IV in the patient's arm, and the MRI table is returned to the original position inside the magnet. The previously prepared cine and first-pass perfusion scans are started using the start button located on the magnet 808; stress function is executed first, followed by stress perfusion 810. The time from end of exercise to start of imaging (Tstart) is recorded. A member of the medical team starts the injection protocol as soon as an audible change from the cine pulse sequence to the first-pass pulse sequence is detected. The patient remains inside the magnet bore for approximately 90 seconds for stress imaging.

Following treadmill exercise, MRI scans are executed to evaluate cardiac function and myocardial perfusion at peak stress. In an example embodiment of the present invention, cardiac function is evaluated using a real-time steady-state free precession (SSFP) pulse sequence with TR/TE of 2.3/1.0 msec and Temporal Sensitivity Encoding (TSENSE) acceleration factor of 3. Five slices are acquired in the short axis (SAX) direction, and one slice each in horizontal (HLA) and vertical (VLA) long axis directions. Temporal resolution of 57 msec and spatial resolution of 3.0 mm×3.8 mm×8 mm may be achieved with no breath-hold and no ECG gating. Each slice position is scanned for approximately two seconds depicting three or more cardiac cycles, depending on heart-rate. Thus, cine images depicting three or more cardiac cycles in each of seven slice locations including short-axis and long-axis views may be acquired in approximately 14 seconds at peak stress. Other data acquisition methods may be used such as improved array coils to accelerate scanning, scanning more slices, or scanning each slice for more heartbeats, or using segmented k-space acquisition methods to improve temporal resolution even further.

In an example embodiment of the present invention, immediately following the acquisition of cardiac function images, first-pass cardiac perfusion images are obtained during intravenous infusion of a contrast agent of 0.1 mmol/kg gadolinium-DTPA at a rate of 4 mL/s. Other doses or rates may be used. A gradient-echo echo-planar (GRE-EPI) imaging sequence with TR/TE of 5.8/1.2 msec and TSENSE acceleration rate of 2 is used to acquire three short-axis slices each cardiac cycle. A saturation recovery time of 30 msec may be used and an acquisition time per slice of 70 msec (96×160 matrix, 3.0 mm×2.4 mm×10 mm resolution). These sequence parameters appear to be optimal, but there are many more options that are feasible.

Other imaging options that may be used in conjunction with the present invention include: cine only covering more slices and views; perfusion only; cine followed by perfusion; perfusion followed by cine; real-time blood flow velocity mapping; real-time myocardial velocity mapping; real-time cardiac tagging for myocardial strain measurement; real-time displacement encoded stimulated echo (DENSE) for myocardial strain measurement; NMR spectroscopy measurement of myocardial metabolism at peak stress; and NMR spectroscopy measurement of skeletal muscle metabolism at peak stress.

Following imaging at peak stress, the patient table is removed from the magnet bore 812 and diagnostic 12-lead ECG and blood pressure monitoring is performed during the supine recovery period lasting approximately 6-10 minutes. Following this recovery period, the patient is moved again into the magnet bore for additional imaging. Resting cardiac function images and resting first-pass perfusion images are acquired 816 are acquired using the methods previously described. After another ten minutes to allow the contrast agent to reach equilibrium, delayed myocardial enhancement (DME) 818 images are acquired to detect any regions of myocardial infarction or fibrosis. Additional scans may be performed to evaluate valve function, diastolic dysfunction, atrial function, size and compliance of the aorta, and a variety of other common cardiovascular MRI techniques.

During the test, a supervising cardiologist may review interim findings, particularly if they warrant termination of exercise such as severe ischemic ECG changes accompanied by worrisome symptoms. Upon completion of the test, the supervising cardiologist assimilates all of the information including the patient's history, any symptoms recorded during exercise, ECG tracings recorded before/during/after exercise, and the CMR images. Software that displays all the images in a format suitable for rapid review and comparison is used. A comprehensive interpretation of the test results may include assessment of the patients exercise capacity, symptoms and their time of onset as well as mode of resolution, ECG changes, and stress-induced contractile and perfusion response. In addition, CMR allows direct visualization of scarred myocardium that can be incorporated into both segmental and patient-level interpretations of normal/no ischemia, fixed infarction, or ischemic response to stress.

A detailed list of features of the present invention and related advantages are summarized in Table 3.

TABLE 3
Features and Advantages of Invention

| Feature | Advantages |
|---|---|
| Water-based Hydraulic Drive System for | water is safe hydraulic fluid with no risk of combustion |
| Treadmill | or harmful spillage |
| | tap water in plentiful supply |
| | no need to store hydraulic fluids |
| | allows MRI-compatible treadmill to feel similar to |
| | standard treadmills |
| | simple and accessible design allows for easy |
| | maintenance |
| Water-based Hydraulic Elevation System | allows treadmill to safely incline within MRI suite |
| for Treadmill | water is safe hydraulic fluid with no risk of combustion |
| | or harmful spillage |
| | tap water in plentiful supply |
| | no need to store hydraulic fluids |
| | allows MRI-compatible treadmill to feel similar to |
| | standard treadmills |
| Treadmill Control System | allows treadmill run independently through standard |
| | PC as well as with leading treadmill stress testing |
| | software |
| MR Compatibility and Safety | Allows treadmill to be used safely within MRI imaging |
| | suite with no effect on image quality |
| Lift system for treadmill to facilitate | allows treadmill to be raised or lowered to allow easy |
| transfers | transfer of patient from treadmill to MRI table |
| ECG system that is compatible - PC | allows exercise stress test to be performed adjacent |
| outside scan room | to MRI unit |
| Vacuum bags for rapid and accurate | allows rapid acquisition of stress cardiac MR images |
| patient repositioning | that match resting acquisition planes |
| | immediate repositioning enables use of previously |
| | defined image planes, saving considerable time |
| | between exercise and imaging |
| Staff and equipment in same room | eliminates need to transfer patient, personnel, and |
| | monitoring equipment from one room to another |
| | feasible for routine clinical use |
| | replicates stress-echo lab |
| | reduces number of staff required to safely execute |
| | stress study |
| | provides maximum patient privacy |
| Imaging software to optimize at high heart | allows imaging of patients immediately post-stress at |
| rate - heavy breathing | peak heart rates without need for breath holding |

Currently, nearly 10 million cardiovascular stress imaging studies performed annually using echocardiography and nuclear scintigraphy. The present invention allows the superior imaging provided by MRI to be used for cardiovascular stress imaging studies. The MRI-compatible treadmill system of the present invention supports the use of MRI which provides a diagnostic advantage over current echocardiography and nuclear scintigraphy. The present invention allows rapid acquisition of MRI images following exercise to more accurately diagnosis cardiovascular disease while increasing patient safety by minimizing the travel required between exercise equipment and the MRI scanner table.

While certain embodiment(s) of the present invention have been described in detail above, the scope of the invention is not to be considered limited by such disclosure, and modifications are possible without departing from the spirit of the invention as evidenced by the following claims:

What is claimed is:

1. A method for performing a Cardiac Magnetic Resonance (CMR) imaging examination following treadmill exercise stress comprising:
(a) positioning in an MRI examination room a non-ferromagnetic treadmill, said treadmill comprising a programmable component;
(b) providing program instructions to said treadmill for execution in said programmable component to perform an exercise protocol at said treadmill causing a patient to achieve cardiovascular stress;
(c) transferring said patient from said treadmill to an MRI examination table after said patient achieves cardiovascular stress;
(d) acquiring from said MRI scanner, magnetic resonance cardiac imaging data using real time imaging techniques that eliminate breath holding requirements; and
(e) analyzing said cardiac image data for said patient to assess presence of cardiovascular disease.

2. The method of claim 1 wherein acquiring from said MRI scanner magnetic resonance cardiac imaging data comprises acquiring said imaging data within 60 seconds after said patient achieves cardiovascular stress.

3. The method of claim 1 wherein acquiring from said MRI scanner magnetic resonance cardiac imaging data comprises acquiring images selected from the group consisting of:
(i) cardiac function images;
(ii) myocardial perfusion images;
(iii) myocardial enhancement images;
(iv) quantitative blood flow velocity images;
(v) myocardial tissue velocity images;
(vi) cardiac tagging for myocardial strain measurement images;
(vii) real-time displacement encoded stimulated echo (DENSE) for myocardial strain measurement images;
(viii) NMR spectroscopy measurement of myocardial metabolism; and
(ix) NMR spectroscopy measurement of skeletal muscle metabolism under stress.

4. The method of claim 1 further comprising connecting said patient to equipment for monitoring physiological parameters.

5. The method of claim 1 wherein positioning a non-ferromagnetic treadmill in proximity to a MRI scanner comprises positioning said treadmill directly adjacent to said MRI examination table.

6. The method of claim 1 wherein said non-ferromagnetic treadmill comprises a hydraulic motor.

7. The method of claim 1 wherein providing program instructions to said treadmill to perform an exercise protocol comprises providing program instructions to control a speed and an elevation of said treadmill.

8. A system for performing a Cardiac Magnetic Resonance (CMR) imaging examination following treadmill exercise stress comprising:
a non-ferromagnetic treadmill positioned in an MRI examination room in proximity to a MRI scanner, said treadmill comprising a programmable component;
a treadmill control computer in communication with said programmable component to perform an exercise protocol at said treadmill causing a patient to reach cardiovascular stress;
a MRI examination table in said MRI examination room for receiving said patient from said treadmill after said patient reaches cardiovascular stress;
a MRI scanner for acquiring cardiac imaging data for said patient using real time imaging techniques that eliminate breath holding requirements; and
a scanner computer for receiving said cardiac imaging data for said patient and analyzing said cardiac image data for said patient to assess presence of cardiovascular disease.

9. The system of claim 8 wherein said MRI scanner magnetic resonance cardiac imaging data comprises images selected from the group consisting of:
(i) cardiac function images;
(ii) myocardial perfusion images;
(iii) myocardial enhancement images;
(iv) quantitative blood flow velocity images;
(v) myocardial tissue velocity images;
(vi) cardiac tagging for myocardial strain measurement images;
(vii) real-time displacement encoded stimulated echo (DENSE) for myocardial strain measurement images;
(viii) NMR spectroscopy measurement of myocardial metabolism; and
(ix) NMR spectroscopy measurement of skeletal muscle metabolism under stress.

10. The system of claim 8 further comprising monitoring equipment connected to said patient for monitoring physiological parameters

11. The system of claim 8 wherein said non-ferromagnetic treadmill comprises a hydraulic motor that is driven by a hydraulic pump connected to said hydraulic motor by hydraulic hoses.

12. The system of claim 8 wherein said exercise protocol instructions comprise instructions to control a speed and an elevation of said treadmill.

13. The system of claim 8 wherein said treadmill further comprises an optical sensor to monitor the speed of said treadmill and to send a fiber optic cable signal to said treadmill control computer.

14. The system of claim 8 wherein said treadmill further comprises an optical sensor to monitor the elevation of said treadmill and to send a fiber optic cable signal to said treadmill control computer.

15. A treadmill comprising:
a support; and
a belt rotatably associated with said support;
wherein said treadmill is comprised of non-ferromagnetic material such that said treadmill is suitable for use in a MRI examination room in close proximity to a magnetic resonance imaging system comprising a scan table for positioning a patient inside a magnet bore.

16. The treadmill of claim 15 wherein a material for said support is selected from the group consisting of stainless steel and aluminum.

17. The treadmill of claim 15 wherein said support comprises at least one drive roller adapted to cause rotation of said belt.

18. The treadmill of claim 15 further comprising a programmable component adapted to execute at least one exercise protocol instruction.

19. The treadmill of claim 15 further comprising a programmable component adapted to execute at least one instruction to control a speed or an elevation of said treadmill.

20. The treadmill of claim 15 further comprising a hydraulic motor adapted to cause rotation of said belt.

21. The treadmill of claim 15 further comprising an optical sensor adapted to monitor a speed of rotation of said belt.

22. The treadmill of claim 15 further comprising an optical sensor adapted to monitor an elevation of said treadmill.

23. The treadmill of claim 15 further comprising a hydraulic cylinder adapted to adjust an elevation of said treadmill.

24. The treadmill of claim 15 further comprising:
a programmable component adapted to execute at least one exercise protocol instruction;
wherein said support comprises at least one drive roller such that said belt is adapted to be rotated about said at least one drive roller.

25. The treadmill of claim 15 further comprising:
a programmable component adapted to execute at least one exercise protocol instruction;
a hydraulic motor adapted to cause rotation of said belt; and
a hydraulic cylinder adapted to adjust an elevation of said treadmill;
wherein said support comprises at least one drive roller in association with said hydraulic motor such that said at least one drive roller is adapted to cause rotation of said belt.
